(19) United States
(12) Patent Application Publication
(10) Pub. No.: US 2011/0268850 A1
(43) Pub. Date: Nov. 3, 2011

(54) MODIFIED ATMOSPHERE PACKAGING GAS, METHOD FOR NON-THERMAL PLASMA TREATMENT OF ARTICLE, AND ARTICLE OF MANUFACTURE FOR USE THEREIN

(76) Inventors: Vashui Rasanayagam, Bear, DE (US); Meenakshi Sundaram, Newark, DE (US)

(21) Appl. No.: 12/772,169

(22) Filed: Apr. 30, 2010

(51) Int. Cl. C01B 31/20; B65B 31/02; B65B 25/00; B65D 81/24; A23B 4/015; A23L 1/31

(52) U.S. Cl. 426/129; 252/372; 426/106; 426/392; 206/210; 53/432

(57) ABSTRACT

A foodstuff or medical device is subjected to a treatment with non-thermal plasma and ozone generated from a modified atmosphere packaging gas comprising a mixture of CO₂ and O₂, wherein a vol/vol ratio of O₂ and CO₂ ranges from 9:1 to 1.5:1.

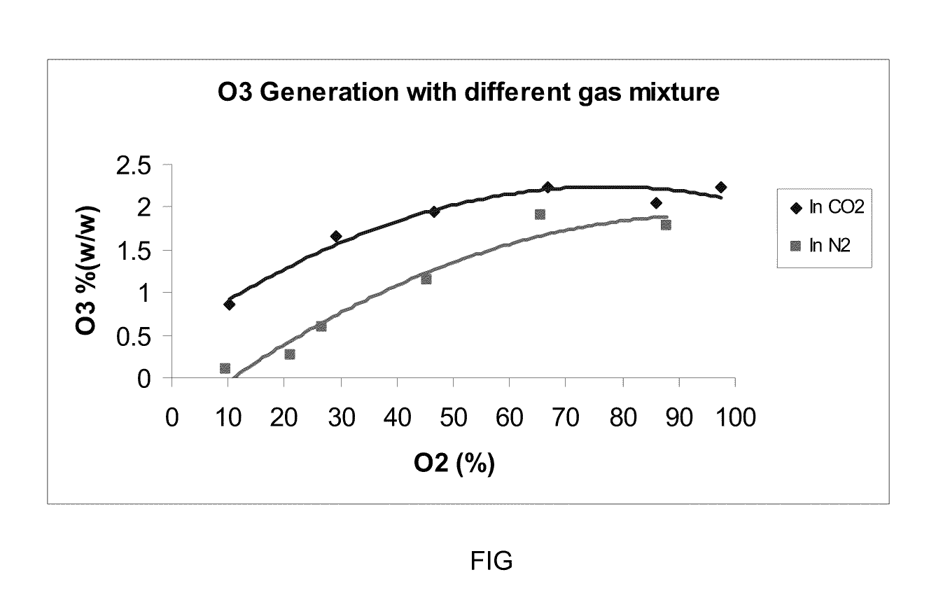

### CROSS-REFERENCE TO RELATED APPLICATIONS

None.

### BACKGROUND

Ozone is a known antimicrobial agent which approved by the U.S. Food & Drug Administration for direct food application. It is generally generated onsite by the corona discharge method using oxygen or air as a feed gas. Several attempts have been made to use ozone as part of a modified atmosphere to reduce the microbial population in packaged food. However, it was found that the amount of ozone needed to sufficiently reduce the microbial population, often had a negative effect upon the quality of the food, such as a bad odor or flavor. Ozone is also a reactive by-product from the plasma ionization of oxygen. Recently, Purdue University developed a method to generate plasma inside a sealed package containing food. Paul A. Klockow, Kevin M. Keener, “Safety and quality assessment of packaged spinach treated with a novel ozone-generation system”, LWT Food Science and Technology, Vol 42, Issue 6, July 2009, pp 1047-1053. The technology can be adopted to generate ozone from air or oxygen inside the package.

One of the current limitations of using air or oxygen in non-thermal plasma/ozone treatment is the process time. In some instances, the process time is greater than 3 minutes, when a more industrially practical process time is probably less than 30 seconds. A limitation of using pure oxygen in non-thermal plasma/ozone treatment is the deleterious impact the ozone can have upon the flavor or aroma of a foodstuff.

Thus, there is a need for an improved method and system that can achieve a shorter process time.

### SUMMARY

There is provided a modified atmosphere packaging gas, comprising a mixture of CO₂ and O₂, wherein a vol/vol ratio of O₂ and CO₂ ranges from 9:1 to 1.5:1.

There is also provided an article of manufacture, comprising a sealed package containing an object and the above modified atmosphere packaging gas, wherein the object is a foodstuff or a medical device.

There is also provided a method of providing a non-thermal plasma and ozone treatment to an article that includes the following steps. The above modified atmosphere packaging gas is subjected to electrical field conditions sufficient for generating a non-thermal plasma and ozone therefrom. An article is placed in an ambient atmosphere of the generated non-thermal plasma and ozone.

The modified atmosphere packaging gas, article or manufacture, and method may include one or more of the following aspects:

the modified atmosphere comprises a mixture of 10-70% of a non-N₂ inert gas and 90-30% of the CO₂ and O₂ mixture.

the modified atmosphere essentially consists of 10-70% of a non-N₂ inert gas and 90-30% of the CO₂ and O₂ mixture.

the non-N₂ inert gas is Ar.

the non-N₂ inert gas is Xe.

the article is a foodstuff.

the foodstuff is poultry or non-cured pork.

the foodstuff is fish.

the article is a medical device.

the medical device is selected from the group consisting of wherein the medical device is selected from the group consisting of non-metallic surgical instruments, endoscopy kits, metallic surgical instruments, non-metallic surgical implants, and metallic surgical implants.

the non-thermal plasma is generated by applying a high voltage potential to electrodes on opposite sides of the article contained within a sealed package or container made of a dielectric material.

the article and ambient atmosphere are contained within a package or container and the non-thermal plasma and ozone are generated remotely from the package or container and are allowed to flow to an interior of the package or container.

the non-thermal plasma and ozone are generated by passing the modified atmosphere packaging gas of claim 1 through a dielectric tube and a high voltage potential is applied to electrodes disposed on opposite sides of the dielectric tube.

the article is a foodstuff; the package or container is integrated with a food packaging machine; and the non-thermal plasma and ozone are allowed to flow to an interior of the package or container after a vacuum is applied to an interior of the package or container.

the non-thermal plasma and ozone are produced continuously and are temporarily stored in a buffer tank prior to flowing to an interior of the container or package.

the non-thermal plasma and ozone are generated at atmospheric pressure and room temperature.

the modified atmosphere packaging gas has the below proportions of CO₂, O₂, and non-N₂ inert or mixture of non-N₂ inert gases:

| parts | parts | % vol/vol CO₂/O₂ | % vol/vol non-N₂ |
|---|---|---|---|
| CO₂** | O₂*** | mixture | inert gas* |
| 1.0-1.5 | 9.0-8.5 | 100 | 0 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 90-85 | 10-15 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 85-80 | 15-20 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 80-75 | 20-25 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 75-70 | 25-30 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 70-65 | 30-35 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 65-60 | 35-40 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 60-55 | 40-45 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 55-50 | 45-50 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 50-45 | 50-55 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 45-40 | 55-60 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 40-35 | 60-65 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |
| 1.0-1.5 | 9.0-8.5 | 35-30 | 65-70 |
| 1.5-2.0 | 8.5-8.0 | | |
| 2.0-2.5 | 8.0-7.5 | | |
| 2.5-3.0 | 7.5-7.0 | | |
| 3.0-3.5 | 7.0-6.5 | | |
| 3.5-4.0 | 6.5-6.0 | | |

*or mixture of non-N₂ inert gases
**relative to O₂
***relative to CO₂

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

The FIGURE is a graph of generated ozone concentration versus oxygen concentration in the modified atmosphere for a variety of binary gas mixtures.

### DESCRIPTION OF PREFERRED EMBODIMENTS

A foodstuff or medical device may be treated with ozone and a non-thermal plasma generated from a modified atmosphere in order to reduce pathogens present thereupon. In the case of foodstuffs, the treatment may also be used to extend the shelf-life of the foodstuff. In the case of medical devices, the treatment may be performed to a degree such that the medical device is considered sterilized.

The modified atmosphere may be a binary gas mixture containing 1-4 parts CO₂ and 9-6 parts O₂. The modified atmosphere may also be a ternary gas mixture containing 10-70 vol/vol % of a non-N₂ inert gas and 90-30 vol/vol % of a gas mixture of 1-4 parts CO₂ and 9-6 parts O₂. The non-N₂ inert gas may be a quantum gas such as He or a noble gas such as Ar or Xe or a mixture of any two or more of He, Ar, and Xe. Particular combinations of CO₂, O₂, and optional non-N₂ inert gases are listed above.

The non-thermal plasma and ozone can be generated inside a sealed package or container, where the article (foodstuff or medical device) is placed. The material of the package or container may be a polymeric bag, glass container, or any material having dielectric properties.

The non-thermal plasma and ozone can be generated within the package or container with placement of electrodes on opposite sides of the package or container. A low power, high voltage is applied to the electrodes to generate the non-thermal plasma and ozone within the package or container which may be sealed. The non-thermal plasma and ozone can alternatively be generated using any one of a variety of non-thermal plasma generation techniques known in the art. The non-thermal plasma and ozone may alternatively be generated remotely from the package or container and allowed to flow into an interior of the package or container in which case the modified atmosphere packaging gas is allowed to flow through a dielectric tube around which the high voltage electrodes are instead placed. In the case of remote generation, the non-thermal plasma and ozone may be allowed to flow into the interior of the package or container simultaneous with application of a vacuum to the package or container within a food packaging machine. In the case or remote generation, the non-thermal plasma and ozone may also be allowed to be temporarily stored within a buffer tank before being allowed to flow into the container or package.

The non-thermal plasma and ozone are normally generated at atmospheric or close to atmospheric pressure and room temperature. The generation time depends on several parameters such as power, gas mixture being ionized, electrode thickness, dielectric properties of the package or container when the non-thermal plasma and ozone are generated in situ, and the dielectric properties of the non-thermal plasma and ozone generation device when the non-thermal plasma and ozone are generated remote from the package or container.

The generation time is defined as the time needed to ionize the gas mixture to generate the desired amount of stable reactive molecules (in this case it is O₃ and CO). The O₂/CO₂ gas mixture reduces plasma generation time considerably for a given plasma set-up.

We have found that the selectivity of Ar or Xe is better than N₂ in terms of increasing the ozone concentration. Ar & Xe not only increase the concentration of ozone, but helps to increase the electrode gap by enhancing the creation of the non-thermal plasma. The method of using gas molecules to increase the ozone concentration, decreases the plasma ionization time and thus makes the technology easier to scale-up.

When the article to be treated with ozone is a foodstuff, the modified atmosphere is especially applicable to pale or white foods such as poultry, uncured pork, or white-fleshed fish. When the article to be treated with ozone is a medical device, the modified atmosphere is especially applicable to non-oxygen sensitive products needing sterilization.

This is because ozone treatment can sometimes bleach color. The gas mixture containing O₂, CO₂ (either CO₂/O₂ mixture or as 3-gas mixture with noble gas) can be subjected to plasma ionization to treat food products such as white meat (chicken, turkey), other non-oxygen sensitive products (e.g. white fish) or medical devices, consumer products (disposable pipettes, containers etc) that needs sterilization

Without being bound by any particular theory, it is believed that the CO generated from the CO2 present in the modified gas atmosphere moderates the sometimes deleterious effect of ozone upon flavors and aromas in the foodstuff to be treated. Thus, a desired ozone level for reduction of pathogens can be generated without the often-associated undesirable flavors and aromas.

For sterilization of medical devices or other consumer products, the ozone concentration need not be limited to 1%. Rather, the ozone concentration is instead driven by the particular type of pathogen of concern as well as the residual ozone concentration that is tolerable in the container or package when open by a user.

### EXAMPLES

Non-thermal plasmas were generated within plastic bags for different binary gas mixtures containing varying levels of CO₂ and O₂ and different binary gas mixtures containing varying levels of N₂ and O₂. Each bag (1 gallon Ziploc brand bags) was filled with 4.5 liters of the gas mixture being tested. The filled bag was placed between two electrodes separated by a gap of 10 mm. The electrodes were formed from coils of wire wound around a dielectric base to provide a treatment area of 51 cm² (8.5 cm by 6 cm). A voltage potential of about 12 kV was applied to the electrodes with a DC voltage generator operated at 55 watts. In each case, a non-thermal plasma was generated for 5 minutes. At the expiration of the plasma generation period, a gas sample was withdrawn from the bag and immediately analyzed with an ozone analyzer as well as by FTIR.

The FIG shows the amount of ozone generated within the bags versus the oxygen content for both types of binary gas mixtures. As clearly shown by the FIG, the inclusion of CO₂ in the gas atmosphere greatly increases the amount of ozone generated.

Performance of the invention yields significant advantages. In the case where it is desirable to package the foodstuff or medical device with relatively lower oxygen content, a desired ozone level is still possible by including CO₂ in the modified atmosphere. Inclusion of a noble or quantum gas (selected as the non-N2 inert gas) such as Ar, Kr (noble gases), or He (quantum gas) within the modified atmosphere enhances plasma generation such that the electrodes may be separated by a much greater inter-electrode distance or gap. Thus, thicker containers or packages may be inserted between the electrodes that might not otherwise fit when the modified atmosphere does not contain such a gas. Additionally, inclusion of such a gas also enhances ozone generation. Thus, when the noble or quantum gas is included to allow greater separation between the electrodes, the amount of ozone being generated should not be deleteriously impacted.

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

What is claimed is:

1. A modified atmosphere packaging gas, comprising a mixture of CO₂ and O₂, wherein a vol/vol ratio of O₂ and CO₂ ranges from 9:1 to 1.5:1.

2. The modified atmosphere packaging gas of claim 1, wherein the modified atmosphere comprises a mixture of 10-70% of a non-N₂ inert gas and 90-30% of the CO₂ and O₂ mixture.

3. The modified atmosphere packaging gas of claim 1, wherein the modified atmosphere essentially consists of 10-70% of a non-N₂ inert gas and 90-30% of the CO₂ and O₂ mixture.

4. The modified atmosphere packaging gas of claim 1, wherein the non-N₂ inert gas is Ar.

5. The modified atmosphere packaging gas of claim 1, wherein the non-N₂ inert gas is Xe.

6. An article of manufacture, comprising a sealed package containing an object and the modified atmosphere packaging gas of claim 1, wherein the object is a foodstuff or a medical device.

7. The article of manufacture of claim 6, wherein the article is a foodstuff.

8. The modified atmosphere packaging gas of claim 7, wherein the foodstuff is poultry or non-cured pork.

9. The article of manufacture of claim 7, wherein the foodstuff is fish.

10. The article of manufacture of claim 6, wherein the article is a medical device.

11. The article of manufacture of claim 10, wherein the medical device is selected from the group consisting of non-metallic surgical instruments, endoscopy kits, metallic surgical instruments, non-metallic surgical implants, and metallic surgical implants.

12. A method of providing a non-thermal plasma and ozone treatment to an article, comprising the steps of:
subjecting the modified atmosphere packaging gas of claim 1 to electrical field conditions sufficient for generating a non-thermal plasma and ozone therefrom; and
placing an article in an ambient atmosphere of the generated non-thermal plasma and ozone.

13. The method of claim 12, wherein the non-thermal plasma is generated by applying a high voltage potential to electrodes on opposite sides of the article contained within a sealed package or container made of a dielectric material.

14. The method of claim 12, wherein:
the article and ambient atmosphere are contained within a package or container; and
the non-thermal plasma and ozone are generated remotely from the package or container and are allowed to flow to an interior of the package or container.

15. The method of claim 14, wherein:
the non-thermal plasma and ozone are generated by passing the modified atmosphere packaging gas of claim 1 through a dielectric tube; and
a high voltage potential is applied to electrodes disposed on opposite sides of the dielectric tube.

16. The method of claim 14, wherein:
the article is a foodstuff;
the package or container is integrated with a food packaging machine; and
the non-thermal plasma and ozone are allowed to flow to an interior of the package or container after a vacuum is applied to an interior of the package or container.

17. The method of claim 14, wherein the non-thermal plasma and ozone are produced continuously and are temporarily stored in a buffer tank prior to flowing to an interior of the container or package.

18. The method of claim 12, wherein the non-thermal plasma and ozone are generated at atmospheric pressure and room temperature.
